(19) United States
(12) Patent Application Publication
(10) Pub. No.: US 2021/0063322 A1
(43) Pub. Date: Mar. 4, 2021

(54) GRAIN MILDEW DETECTION METHOD AND DEVICE BASED ON WIFI APPARATUS

(71) Applicant: Henan University of Technology, Zhengzhou (CN)

(72) Inventors: Weidong Yang, Zhengzhou (CN); Pengming Hu, Zhengzhou (CN); Yuan Zhang, Zhengzhou (CN); Wei Wei, Zhengzhou (CN); Zhi Li, Zhengzhou (CN); Yao Qin, Zhengzhou (CN); Chunhua Zhu, Zhengzhou (CN); Yuying Jiang, Zhengzhou (CN); Wenshuai Zhang, Zhengzhou (CN); Xingxing Chen, Zhengzhou (CN)

(21) Appl. No.: 17/010,164

(22) Filed: Sep. 2, 2020

(30) Foreign Application Priority Data
Sep. 3, 2019 (CN) 201910829383.6

(51) Int. Cl. G01N 22/00; G01N 33/02; H04W 24/08; G06N 3/08

(52) U.S. Cl. CPC G01N 22/00; G01N 33/02; H04W 84/12; G06N 3/08; H04W 24/08

(57) ABSTRACT

A grain mildew detection method and device based on a WiFi apparatus. The method includes the following steps: acquiring a WiFi signal which passes through a grain region, extracting channel state information (CSI) amplitude data from the WiFi signal, and acquiring grain statuses corresponding to the CSI amplitude data; establishing a neural network model, and training the neural network model by using the acquired CSI amplitude data and the grain statuses corresponding to the CSI amplitude data, to obtain an amplitude-status relationship model; and acquiring a WiFi signal which passes through a region in which grain to be detected is located, extracting CSI amplitude data from the WiFi signal which passes through the region in which the grain to be detected is located, and inputting the CSI amplitude data into the amplitude-status relationship model, to obtain a grain status of the grain to be detected.

### CROSS-REFERENCE TO RELATED APPLICATIONS

This Application claims priority to Chinese Application No. 201910829383.6 filed on Sep. 3, 2019, the content of which is incorporated herein by reference in its entirety.

### TECHNICAL FIELD

The present invention relates to the field of grain mildew detection technologies, and in particular, to a grain mildew detection method and device based on a WiFi apparatus.

### BACKGROUND

Grain (for example, wheat and rice) mildew can lead to pollution of stored cereals, loss of nutrients, and foodborne diseases in humans Microbial and environmental factors are mainly responsible for grain mildew. Mildew is usually caused by the microbes in wheat granules during harvesting and by the granary microorganisms during storage. On the other hand, grain mildew is also affected by granary type, temperature, humidity, and other environmental factors. In the early stage of grain mildew, if timely measures are taken, the grain will still be of use value. When the grain has been completely mildewed, it will lose the use value and should be destroyed as soon as possible to avoid causing human diseases. A real-time, non-destructive, and low-cost grain mildew detection system can be highly useful to ensure high safety of grain storage.

Due to the lack of professional knowledge and the high cost of testing equipment, many farmers and distributors cannot timely test the status of grain. Rapid detection of mildew in grain can help farmers, distributors, and retailers to achieve more efficient and safer food storage, and thus to reduce food waste and cost.

It is a great challenge to detect mildew in grain quickly and at a low-cost. At present, detection of grain mildew mainly depends on manual detection. In fact, the degree of grain mildew is judged based on visual inspection and the olfactory experience of the inspector. The manual approach is time-consuming, error-prone, and not much helpful to quickly detect grain mildew. In order to improve detection efficiency, costly sensors, such as an electronic nose sensor and a near-infrared spectroscopy, may be used to detect grain mildew. However, these sensors are required to be laid in a large area in a detection region so that wheat in the whole detection region can be detected, which undoubtedly increases the detection cost and impedes widespread application of these sensors.

### SUMMARY

The present invention provides a grain mildew detection method and device based on a WiFi apparatus, so as to solve the problem of high cost caused by the use of the electronic nose sensors and near-infrared spectroscopy to detect grain mildew.

To solve the foregoing technical problem, the present invention adopts the following technical solutions and achieves subsequently described advantageous effects:

The present invention provides a grain mildew detection method based on a WiFi apparatus, including the following steps: acquiring a WiFi signal which passes through a grain region, extracting channel state information (CSI) amplitude data from the WiFi signal, and acquiring grain statuses corresponding to the CSI amplitude data, where the grain statuses include a normal status and a mildew status; establishing a neural network model, and training the neural network model by using the acquired CSI amplitude data and the grain statuses corresponding to the CSI amplitude data, to obtain an amplitude-status relationship model; and acquiring a WiFi signal which passes through a region in which grain to be detected is located, extracting CSI amplitude data from the WiFi signal which passes through the region in which the grain to be detected is located, and inputting the CSI amplitude data into the amplitude-status relationship model, to obtain a grain status of the grain to be detected.

Advantageous effects are as follows: When a WiFi signal passes through grain, a change in the grain mildew status causes significant and measurable changes of CSI amplitude data in the WiFi signal. The present invention depends on this principle and establishes a neural network model, so as to detect whether the grain is mildewed. The present invention can realize grain mildew detection by using an existing WiFi apparatus and software algorithm, and can incessantly detect a grain mildew status for a long time, without the need to use expensive sensors, thus having a low detection cost and facilitating practical application. Moreover, the method employs a trained amplitude-status relationship model which is simple and effective, achieving high real-time performance Thus, farmers and dealers can efficiently and rapidly find out whether the grain is mildewed, so as to reduce grain waste and cost.

As a further improvement to the method, in order to accurately detect a grain mildew status, the mildew status includes an initial stage of mildew and complete mildew.

As a further improvement to the method, the neural network model is a radial basis function (RBF) neural network model.

As a further improvement to the method, in order to select CSI amplitude data from high-sensitivity subcarriers to improve the accuracy of grain mildew detection, when the neural network model is trained, the method further includes a step of subcarrier selection on the acquired CSI amplitude data: calculating a mean absolute deviation of CSI amplitude data of each subcarrier, determining subcarriers corresponding to CSI amplitude data of which the mean absolute deviations are greater than a set deviation, and selecting CSI amplitude data from the determined subcarriers to train the neural network model.

As a further improvement to the method, in order to eliminate outliers and noise to improve the accuracy of grain mildew detection, before the step of subcarrier selection on the acquired CSI amplitude data, the method further includes a step of filtering pre-processing for the acquired CSI amplitude data: performing outlier elimination from the acquired CSI amplitude data, and/or performing noise suppression for the acquired CSI amplitude data.

As a further improvement to the method, in order to speed up the grain mildew detection and improve its accuracy, the method further includes a step of normalization processing on the CSI amplitude data obtained after the subcarrier selection.

As a further improvement to the method, the outlier elimination is filtering processing with a Hampel filter.

As a further improvement to the method, the noise suppression is filtering processing with a Butterworth filter.

As a further improvement to the method, during use of the RBF neural network model, the number of hidden neurons in an RBF function is determined by using a clustering algorithm, and the number of clusters equals the number of the hidden neurons.

The present invention further provides a grain mildew detection device based on a WiFi apparatus, where the device includes a memory and a processor, and the processor is used to execute instructions stored in the memory so as to implement the above-described grain mildew detection method based on a WiFi apparatus, thus achieving the same effects as the method.

### BRIEF DESCRIPTION OF DRAWINGS

FIG. 1 is a schematic diagram of original CSI amplitude values acquired from wheat piles in three mildew statuses in a method embodiment of the present invention;

FIG. 2 is an architecture diagram of a MiFi system corresponding to a grain mildew detection method in the method embodiment of the present invention;

FIG. 3 is a schematic diagram of CSI data acquired from the 20^{th} subcarrier before and after calibration in the method embodiment of the present invention;

FIG. 4 is a schematic diagram of spectrums of CSI data from the 20^{th} subcarrier for the three mildew statuses in the method embodiment of the present invention;

FIG. 5 is a schematic diagram of calibrated CSI amplitudes on all subcarriers in the method embodiment of the present invention, which are used to select the most sensitive subcarriers;

FIG. 6 is a result graph showing the accuracy of wheat mildew detection in line-of-sight (LOS) and non-line-of-sight (NLOS) scenarios in the method embodiment of the present invention;

FIG. 7 is a result graph showing an average detection accuracy of different antennas in the LOS and NLOS scenarios in the method embodiment of the present invention; and

FIG. 8 is a result graph showing an average detection accuracy at different transmitter-to-receiver distances in the method embodiment of the present invention.

### DETAILED DESCRIPTION

When grain, for example, wheat, is mildewed, in order to quantify the effect, the concept of dielectric constant may be used to indicate the change of a wheat mildew status. A complex relative permittivity ε* of a material in a frequency domain may be expressed as follows:

ε*=ε′−j″ (1)

where the real part ε′ is the dielectric constant, which indicates an ability of the material to store energy in the frequency domain of an electric field; and the imaginary part ε″ is a dielectric loss factor, which usually indicates the ability of a material to consume electrical energy, thus affecting the attenuation and absorption of WiFi signals.

When the WiFi signal passes through the wheat, the intensity of the electric field changes with a distance to the surface of the wheat. Such an effect can be captured by using an attenuation factor a regarding dielectric properties of grains:

$$α = \frac{2 π}{{λ}_{0}} \sqrt{\frac{{ɛ}^{′}}{2} ( \sqrt{1 + {( \frac{{ɛ}^{″}}{{ɛ}^{′}} )}^{2} - 1} )} \qquad ( 2 )$$

where λ0 is a wavelength of the wireless signal.

The change of the wheat status from normal, to an initial stage of mildew, and finally to complete mildew may cause an increase in wheat temperature and moisture, and humidity in an external environment. These changes may in turn affect the permittivity ε′ and the dielectric loss factor ε″. According to the formula (2), the attenuation factor a may also be changed (as a function of ε′ and ε″) to affect the energy of the electric field. Compared to the normal wheat, the wheat mildew highly affects the energy of the electric field.

In order to quantify such a change in energy, the wheat mildew status is detected by analyzing WIFi-based CSI amplitude information, without the need to use a costly apparatus to measure the permittivity, thus effectively preventing wheat mildew.

By using network interface cards (NICs) of some products having an open-source device driver, CSI samples may be collected from Ns subcarriers, and each sample includes an amplitude and phase of the subcarrier. The collected original data includes the number Nₜₓ of transmitting antennas, the number Nᵣₓ of receiving antennas, a packet transmission frequency f, and CSI data H. The CSI data H is a tensor quantity of Nₜₓ×Nᵣₓ×Nₛ, and is given by the following formula:

H=(Hᵢⱼₖ)_{N}_{ₜₓ}_{×N}_{ᵣₓ}_{×N}_{ₛ} (3)

For a given pair of transmitting and receiving antennas, the kth subcarrier in the data H may be expressed as follows:

Hₖ=|Hₖ|·exp{j∠Hₖ} (4)

where |Hₖ| is the amplitude and ∠Hₖ is the phase.

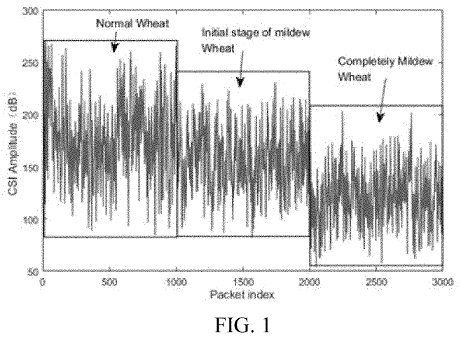

The wheat mildew not only changes the moisture in the whole wheat environment, but also changes the temperature and air humidity of the wheat environment, thus further affecting the electric field. CSI amplitude data of the same wheat pile is collected (as well as a relative position of the wheat pile and a WiFi apparatus). The wheat has three development statuses which are a normal status, an initial stage of mildew, and complete mildew. FIG. 1 shows CSI amplitude data collected under the foregoing three statuses. The abscissa in FIG. 1 indicates received WiFi data packets, and the ordinate indicates the CSI amplitude data (in dB), where “Normal Wheat” refers to wheat in a normal status, “Initial stage of Mildew Wheat” refers to wheat in an initial stage of mildew, and “Completely Mildew Wheat” refers to completely mildewed wheat. It can be seen from FIG. 1 that, when the wheat changes from the normal status to the initial stage of mildew, the CSI amplitude merely slightly changes; while when the wheat is completely mildewed, the CSI amplitude data obviously varies a lot. Therefore, the present invention uses the CSI amplitude data for wheat mildew detection. A wheat mildew detection method by using the CSI amplitude data will be described in detail below.

Method Embodiments

Driven by an existing WiFi-based CSI sensing technology, this embodiment aims to provide a low-cost, contactless, and long-term mold prevention and monitoring method, and thus proposes a grain mildew detection method based on a WiFi apparatus. The following content uses wheat as an example to describe this method. Wheat mildew involves a series of physiological changes inside and outside the wheat. When a WiFi signal passes through the wheat, the change of the wheat mildew status causes significant and measurable changes in the WiFi signal, as recorded by CSI values.

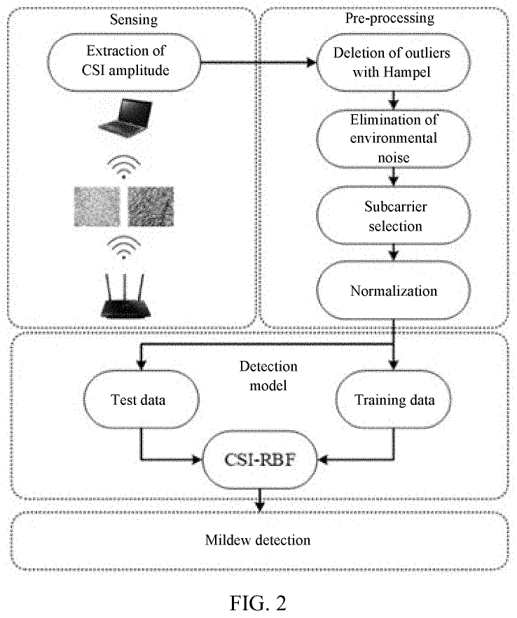

To implement the foregoing method, a hardware construction is arranged as follows: A transmitter for transmitting a WiFi signal to a detection region in which the wheat grows is disposed in the detection region, where the WiFi signal can pass through the wheat. The number of the transmitters is not limited and may be set according to a size of the detection region, so that the WiFi signal is covered in the whole detection region. A data processing terminal is disposed in or out of the detection region, and includes a receiver and a signal processor. The receiver is configured to receive the WiFi signal transmitted by the transmitter and transmit the received WiFi signal to the signal processor. The signal processor processes the signal to determine a wheat status in the detection region. Specifically, a MiFi system architecture (Device-free Wheat Mildew Detection Using Off-the-shelf WiFi Devices) shown in FIG. 2 is designed to show software processing logic inside the signal processor, and includes four modules which are a sensing module, a pre-processing module, a detection modelling module, and a module for mildew detection.

First, the sensing module is used to acquire a WiFi signal which is transmitted by the transmitter and passes through a wheat region, extract CSI amplitude data from the WiFi signal, and acquire wheat statuses corresponding to the CSI amplitude data. The wheat statuses include a normal status, an initial stage of mildew, and complete mildew.

Specifically, the CSI amplitude data can be collected from 56 subcarriers by using the Atheros AR5BHB NIC. For the normal wheat, the CSI amplitude data is directly collected and transmitted by using a WiFi data packet passing through the wheat piles. With regard to the wheat in an initial stage of mildew and the completely mildewed wheat, because a neural network needs a large number of related samples, wheat mildew can be directly cultivated in a laboratory in which the temperature and humidity can be controlled and adjusted, and mildew growth is accelerated in the wheat, so as to acquire a large number of wheat samples in an initial stage of mildew and completely mildewed wheat samples. During the experiment, the temperature is maintained at 30° C. and the air humidity is maintained at 90%. After 2 to 3 days, mold begins to grow on the wheat and samples in an initial stage of mildew are collected, and the completely mildewed samples are acquired on the 8^{th} day. CSI amplitude data is collected by using the mildewed wheat. In this way, three types of CSI amplitude data can be collected for detection and study on the wheat in different mildew stages.

Afterwards, the pre-processing module is used to pre-process the acquired CSI amplitude data, so as to speed up a computational speed of an established neural network model and improve detection accuracy. A specific pre-processing procedure includes four steps, which are outlier elimination with Hampel, environmental noise removal, subcarrier selection, and normalization.

1. Outlier Elimination with Hampel

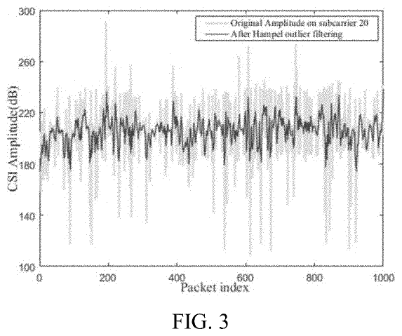

CSI data outliers inevitably occur in the collected CSI amplitude data. For example, as shown in FIG. 3, many peaks and troughs can be seen from the CSI amplitude data collected from the 20^{th} subcarrier. These peak and trough values are the outliers to be eliminated. In the MiFi system architecture, a Hampel filter is used to detect and remove values obviously different from those in a normal CSI amplitude sequence. In FIG. 3, the abscissa indicates received WiFi data packets, and the ordinate indicates the CSI amplitude data (in dB), where “Original Amplitude on subcarrier 20” refers to CSI amplitude data collected from the 20^{th} subcarrier.

Specifically, a Hampel filter with a sliding window is applied in each subcarrier to eliminate the outliers. A CSI amplitude sequence of N samples acquired from the subcarriers is denoted by (X₁, X₂, . . . , X_{N}), where Xᵢ indicates the ith sample in the CSI amplitude sequence acquired from the subcarriers. X′ is let to be a median value in the CSI amplitude sequence. If a Hampel identifier and a median absolute difference (MAD) deviate from a preset threshold, the data point Xᵢ is classified as an outlier:

$$\{ \begin{matrix}\vert {X}_{i} - {X}^{′} \vert > l \cdot R & outlier \\ \vert {X}_{i} - {X}^{′} \vert \leq l \cdot R & normal\end{matrix} \\ i = 1 , 2 , … , N \qquad ( 5 )$$

where l is a pre-defined threshold, and R is the MAD and is defined as follows:

R=1.4286·median{|Xᵢ−X′|, i=1, 2, . . . , N} (6)

where the constant 1.4286 guarantees that an expected value of R equals a standard deviation of normally distributed data.

In FIG. 3, “After Hampel outlier filtering” refers to CSI amplitude data obtained after outliers are eliminated with the Hampel filter. According to the CSI amplitude data from the 20^{th} subcarrier that is calibrated by means of Hampel filtering, it can be learned that the outliers are effectively eliminated.

2. Environmental Noise Removal

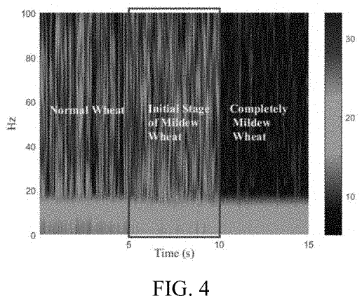

The calibrated CSI data still contains environmental noise. After the outliers are eliminated, the environmental noise still needs to be reduced so as to achieve high detection accuracy. FIG. 4 shows spectrums of CSI data from the 20^{th} subcarrier for the three mildew statuses, where the abscissa indicates the time and the ordinate indicates the frequency. It is learned after observation that the frequency variation caused by mildew wheat over a period of time ranges from 0 Hz to 30 Hz. Therefore, noise, including environmental noise, at other frequencies is suppressed with a Butterworth filter. The Butterworth filter uses a Butterworth function to approximate system functions of a filter. The system functions are defined according to amplitude-frequency characteristics in a passband. A square function of the Butterworth filter in a low pass mode is given by the following formula:

|L(f)|²=(1+(f/f_{c})^{2m})⁻¹ (7)

where m is an order of the filter, f_{c} is a cut-off frequency, and m may be set to 4 and f_{c} may be set to 30 Hz in this MiFi system.

3. Subcarrier Selection

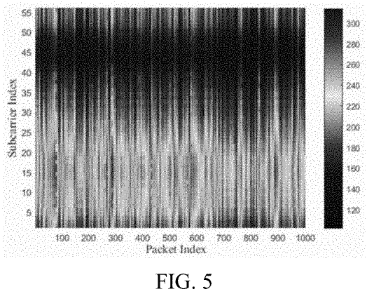

After noise elimination, the CSI amplitude data has components at different low frequencies, and shows different degrees of sensitivity to different mildew statuses of the wheat. A mean absolute deviation of the CSI amplitude data from each subcarrier is used herein to measure the sensitivity of the subcarrier. A larger mean absolute deviation usually indicates higher sensitivity. As shown in FIG. 5, the abscissa indicates WiFi data packets and the ordinate indicates subcarrier indexes. It can be seen from FIG. 5 that subcarriers (among 56 subcarriers) with an index below 35 are more sensitive (as shown by a gray zone in FIG. 5) and more susceptible to wheat mildew. Therefore, in the MiFi system, CSI amplitude data is chosen from these more sensitive subcarriers with an index below 35.

4. Normalization

In order to accelerate computation by the model and improve the detection accuracy, the CSI amplitude data is normalized by means of zero-mean normalization (namely, Z-score normalization). Normalized data V, is calculated by the following formula:

$${V}_{i} = \frac{1}{σ} ( {X}_{i} - \overline{X} ) \qquad ( 8 )$$

where Xᵢ and σ are respectively a mean value and a standard deviation of the CSI amplitude data of the subcarriers.

Afterwards, the detection modelling module is used to establish a neural network model. The normalized CSI amplitude data and wheat statuses corresponding to the CSI amplitude data are respectively used as training data and test data to train the neural network model, to obtain a correspondence between the CSI amplitude data and the wheat statuses, that is, to obtain an amplitude-status relationship model. It should be noted that, the wheat is required to be identical in weight and piling shape during acquisition of the training data and test data.

An RBF neural network model is selected as the established neural network model, and the trained amplitude-status relationship model is referred to as a CSI-RBF neural network model. A K-means clustering algorithm is used to determine the number of hidden neurons in an RBF kernel function.

1. K-Means Clustering Algorithm

The K-means clustering algorithm is widely used for data clustering in many fields, which can be used as an unsupervised learning means to identify parameters of a basis function and determine the number of hidden neurons that is equal to the number of clusters. In the established CSI-RBF model, clustering is performed on the CSI amplitude sequence based on similarity scores, and the similarity scores are calculated according to the amplitude data and the Euclidean distance between cluster mean values. The Euclidean distance (which takes the form of two time sequences, each having a size of N) between two CSI amplitude sequences is given by the following formula:

D(V¹,V²)=\sqrt{(V₁¹−V₁²)²+ . . . +(V_{N}¹−V_{N}²)²} (9)

where V¹ and V² indicate two CSI data streams.

2. CSI-RBF Neural Network Model

An RBF neural network can overcome the shortcomings of slow convergence and local minimum, and has a global approximation capability, thus achieving desired performance in non-linear relationship modeling with fast convergence characteristics. Based on the foregoing advantages, this embodiment uses the RBF neural network to rapidly detect the wheat mildew.

Specifically, the MiFi system uses the RBF neural network to carry out a sorting algorithm. The RBF neural network is basically formed by input neurons, hidden neurons, and output neurons. In the MiFi system, clustering is performed in an input layer, and a CSI amplitude matrix V=(V₁, V₂, . . ., V_{N}) is delivered to F hidden neurons. A hidden layer can map a network input in a non-linear manner, and each hidden neuron is linked to a center and width of each cluster. Multiple activated functions can be applied in the hidden layer, so as to maximize an output accuracy. A used Gaussian function is as follows:

$$θ ( v ) = exp \{ - {( \frac{v - γ}{β} )}^{2} \} \qquad ( 10 )$$

where ν, γ, and β are respectively a pre-determined input vector (namely, the normalized CSI amplitude data), a cluster center vector, and a width (an average distance between the cluster center vector and samples belonging to the corresponding cluster) of the hidden neuron; and γ is a cluster center vector corresponding to a cluster which ν belongs to. It should be noted that, the number of the hidden neurons is equal to the number of clusters, namely, the number of the clusters in the K-mean clustering algorithm.

An output layer uses a linear weighted sum function as an output of the hidden layer. The wheat status can be identified when m=3, and the linear function of the output layer is defined by the following formula:

$${Z}_{m} = {y}_{m} ( w , v ) = {\sum }_{j = 1}^{F} {w}_{jm} \cdot {θ}_{j} ( v ) + b \qquad ( 11 )$$

where Zₘ is the mth output neuron, wⱼₘ is a weight from the jth hidden neuron to the mth output neuron, θⱼ is the Gaussian function in the hidden neurons, and b is a deviation. The CSI amplitude data collected in different mildew statuses is classified into m types. The weight between the hidden layer and the output layer can be easily calculated by means of ordinary least squares (OLS) and linear regression.

A classification matrix for detection of mildew in wheat is calculated as follows by using a combination of linear and non-linear RBF neural network models:

Z=[Z₁, Z₂, . . . , Zₘ] (12)

where m=3, the vector Z₁ is regarded as an output corresponding to normal wheat, the vector Z₂ is regarded as an output corresponding to an initial stage of mildew, and the vector Z₃ is regarded as an output corresponding to complete mildew.

Finally, the module for mildew detection is used to acquire a WiFi signal which passes through a region in which wheat to be detected is located, extract CSI amplitude data from the WiFi signal which passes through the region in which the wheat to be detected is located, and input the CSI amplitude data into the trained CSI-RBF neural network model, to obtain a wheat status of the wheat to be detected. It should be noted that, the wheat to be detected and the wheat trained in the model are required to be identical in weight and piling shape.

A wheat experiment is carried out below to describe feasibility and accuracy of the method of the present invention.

1. Wheat Preparation

Normal wheat and mildewed wheat are separately prepared. The mildewed wheat is prepared in a constant temperature and humidity laboratory and taken out therefrom on the 8^{th} day. The temperature and humidity in the wheat samples are measured. In addition, a moisture content is measured by using a standard drying method.

During the experiment, three different types of wheat samples of the same weight, including normal wheat, wheat in an initial stage of mildew, and completely mildewed wheat, are used to test corresponding mildew conditions. The following table I provides a moisture content, temperature, and humidity of the three different types of wheat samples.

TABLE I
Conditions of wheat samples in the experiment

| | Normal | Initial stage of mildew | Complete mildew |
|---|---|---|---|
| Moisture content | 11.8% | 12.9% | 16.8% |
| Temperature | 17° C. | 20° C. | 30° C. |
| Internal air humidity | 32% | 48% | 77% |

2. MiFi Hardware Structure

Hardware in the experiment includes two Dell PP181 notebook computers fitted with the Atheros AR5BHB NIC (a WLAN card), where one of them is equipped with a single antenna as a transmitter and the other one is equipped with three antennas as a receiver. The two notebook computers both run the Ubuntu Linux 14.04 operating system with the kernel of 4.1.10+32 bits and have 2 GB RAM.

To test the effectiveness of the MiFi system, LOS and NLOS scenarios are separately taken into consideration. In the LOS scenario, the wheat is placed in the middle among the antennas, while in the NLOS scenario, the wheat is not placed in the middle among the antennas. In the two experimental solutions, the transmitter and the receiver are placed at the two ends, and different wheat samples are placed therebetween for acquisition of CSI data.

3. Result of the Experiment

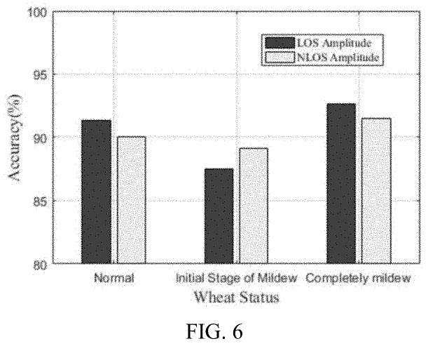

FIG. 6 shows the accuracy of wheat mildew detection in the LOS and NLOS scenarios by using CSI amplitude data, where the abscissa indicates the wheat statuses and the ordinate indicates the accuracy; the dark color corresponds to the LOS scenario and the light color corresponds to the NLOS scenario. In the LOS scenario, it can be learned that the MiFi system can achieve a detection accuracy of above 90% in the cases where the wheat is normal and the wheat is completely mildewed. For the wheat in an initial stage of mildew, the detection accuracy is less than 90% but still reaches 87.5%. An average accuracy in the LOS scenario is 90.48%. In the NLOS scenario, an average accuracy reaches 90.2%. Therefore, the proposed MiFi system is competent enough to detect wheat mildew in both the LOS and NLOS scenarios. The reason is that an impact of the wheat mildew on transmission of the WiFi signal can be precisely captured by CSI amplitude data.

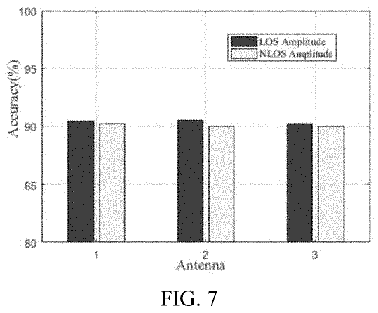

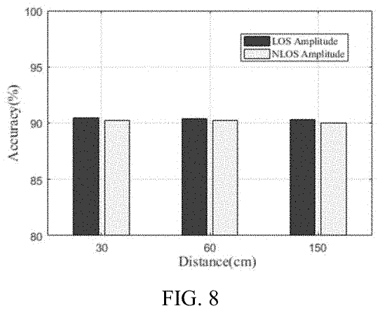

An impact of the configuration of the MiFi system on the detection accuracy is studied below. This experiment focuses on effects brought by different antennas and distances. FIG. 7 shows an average detection accuracy of different antennas of a transmitter that are used in the LOS and NLOS scenarios, where the abscissa indicates the antennas and the ordinate indicates the accuracy; the dark color corresponds to the LOS scenario and the light color corresponds to the NLOS scenario. The result suggests that data acquired with all the three antennas is effective. The average detection accuracy in each of the two scenarios is higher than 90%. FIG. 8 shows an average detection accuracy at different transmitter-to-receiver distances in the LOS and NLOS scenarios, where the abscissa indicates the distance and the ordinate indicates the accuracy; the dark color corresponds to the LOS scenario and the light color corresponds to the NLOS scenario. It can be learned that, at different transmitter-to-receiver distances ranging from 30 cm to 150 cm, the MiFi system maintains a detection accuracy of above 90% all the time.

In this embodiment, an RBF neural network model is selected as the neural network model. As another implementation, another neural network model, for example, a back propagation (BP) neural network, in the prior art may be selected, but has a detection effect not as good as the RBF neural network.

In this embodiment, pre-processing on the acquired CSI amplitude data includes four steps, which are outlier elimination, environmental noise removal, subcarrier selection, and normalization. The four steps are progressive to implement a desired processing manner. First, outliers are eliminated to realize rough filtering; then the environmental noise is eliminated to realize fine filtering; afterwards, the most sensitive subcarriers are selected; and finally normalization is performed. As another implementation, the rough filtering may be skipped and the fine filtering is directly performed; or the normalization processing is omitted; or only the rough filtering is performed and the fine filtering is skipped; or even the pre-processing procedure is wholly skipped; or the like. These manners are all feasible, only that the effect is not as good as that achieved by the method in this embodiment. Moreover, a specific filter used to implement the rough filtering and the fine filtering is not limited herein, as long as a currently used filter can achieve required filtering effects.

In this embodiment, the neural network model has three output results respectively corresponding to three wheat statuses which are a normal status, an initial stage of mildew, and complete mildew. As another implementation, during construction of the neural network model, two output results which respectively indicate normal wheat and mildewed wheat may be set. However, this manner can only roughly determine whether the wheat is mildewed, but cannot output a detection result as accurate as that in the foregoing embodiment.

Device Embodiment

This embodiment provides a grain mildew detection device based on a WiFi apparatus, which includes a memory and a processor. The memory and the processor are directly or indirectly electrically connected to implement data transmission and interaction. The processor may be a general-purpose processor such as a central processing unit (CPU); or may also be a programmable logic device such as a digital signal processor (DSP). The processor is used to execute instructions stored in the memory so as to implement the grain mildew detection method based on a WiFi apparatus that is introduced in the method embodiment. The method has been described in detail in the method embodiment, so the details are not described herein again.

Although the content of the present invention has been described in detail through the aforementioned preferred embodiments, it should be recognized that the above description should not be considered as limiting the present invention. Upon reading the aforementioned content, it will be apparent to those skilled in the art that various modifications and alternations to the present invention can be made. Therefore, the claimed scope of the present invention shall be defined by the appended claims.

What is claimed is:

1. A grain mildew detection method based on a WiFi apparatus, comprising the following steps:
acquiring a WiFi signal which passes through a grain region, extracting channel state information (CSI) amplitude data from the WiFi signal, and acquiring grain statuses corresponding to the CSI amplitude data, wherein the grain statuses comprise a normal status and a mildew status;
establishing a neural network model, and training the neural network model by using the acquired CSI amplitude data and the grain statuses corresponding to the CSI amplitude data, to obtain an amplitude-status relationship model; and
acquiring a WiFi signal which passes through a region in which grain to be detected is located, extracting CSI amplitude data from the WiFi signal which passes through the region in which the grain to be detected is located, and inputting the CSI amplitude data into the amplitude-status relationship model, to obtain a grain status of the grain to be detected.

2. The grain mildew detection method based on a WiFi apparatus according to claim 1, wherein the mildew status comprises an initial stage of mildew and complete mildew.

3. The grain mildew detection method based on a WiFi apparatus according to claim 1, wherein the neural network model is a radial basis function (RBF) neural network model.

4. The grain mildew detection method based on a WiFi apparatus according to claim 1, wherein when the neural network model is trained, the method further comprises a step of subcarrier selection on the acquired CSI amplitude data: calculating a mean absolute deviation of CSI amplitude data of each subcarrier, determining subcarriers corresponding to CSI amplitude data of which the mean absolute deviations are greater than a set deviation, and selecting CSI amplitude data from the determined subcarriers to train the neural network model.

5. The grain mildew detection method based on a WiFi apparatus according to claim 4, wherein before the step of subcarrier selection on the acquired CSI amplitude data, the method further comprises a step of filtering pre-processing for the acquired CSI amplitude data:
performing outlier elimination from the acquired CSI amplitude data, and/or performing noise suppression for the acquired CSI amplitude data.

6. The grain mildew detection method based on a WiFi apparatus according to claim 4, further comprising a step of normalization processing on the CSI amplitude data obtained after the subcarrier selection.

7. The grain mildew detection method based on a WiFi apparatus according to claim 5, further comprising a step of normalization processing on the CSI amplitude data obtained after the subcarrier selection.

8. The grain mildew detection method based on a WiFi apparatus according to claim 5, wherein the outlier elimination is filtering processing with a Hampel filter.

9. The grain mildew detection method based on a WiFi apparatus according to claim 5, wherein the noise suppression is filtering processing with a Butterworth filter.

10. The grain mildew detection method based on a WiFi apparatus according to claim 3, wherein during use of the RBF neural network model, the number of hidden neurons in an RBF function is determined by using a clustering algorithm, and the number of clusters equals the number of the hidden neurons.

11. A grain mildew detection device based on a WiFi apparatus, comprising a memory and a processor, wherein the processor is used to execute instructions stored in the memory so as to implement the grain mildew detection method based on a WiFi apparatus according to claim 1.

12. A grain mildew detection device based on a WiFi apparatus, comprising a memory and a processor, wherein the processor is used to execute instructions stored in the memory so as to implement the grain mildew detection method based on a WiFi apparatus according to claim 2.

13. A grain mildew detection device based on a WiFi apparatus, comprising a memory and a processor, wherein the processor is used to execute instructions stored in the memory so as to implement the grain mildew detection method based on a WiFi apparatus according to claim 3.

14. A grain mildew detection device based on a WiFi apparatus, comprising a memory and a processor, wherein the processor is used to execute instructions stored in the memory so as to implement the grain mildew detection method based on a WiFi apparatus according to claim 4.

15. A grain mildew detection device based on a WiFi apparatus, comprising a memory and a processor, wherein the processor is used to execute instructions stored in the memory so as to implement the grain mildew detection method based on a WiFi apparatus according to claim 5.

16. A grain mildew detection device based on a WiFi apparatus, comprising a memory and a processor, wherein the processor is used to execute instructions stored in the memory so as to implement the grain mildew detection method based on a WiFi apparatus according to claim 6.

17. A grain mildew detection device based on a WiFi apparatus, comprising a memory and a processor, wherein the processor is used to execute instructions stored in the memory so as to implement the grain mildew detection method based on a WiFi apparatus according to claim 7.

18. A grain mildew detection device based on a WiFi apparatus, comprising a memory and a processor, wherein the processor is used to execute instructions stored in the memory so as to implement the grain mildew detection method based on a WiFi apparatus according to claim 8.

19. A grain mildew detection device based on a WiFi apparatus, comprising a memory and a processor, wherein the processor is used to execute instructions stored in the memory so as to implement the grain mildew detection method based on a WiFi apparatus according to claim 9.

20. A grain mildew detection device based on a WiFi apparatus, comprising a memory and a processor, wherein the processor is used to execute instructions stored in the memory so as to implement the grain mildew detection method based on a WiFi apparatus according to claim 10.
